(19) United States
(12) Patent Application Publication
(10) Pub. No.: US 2026/0315246 A1
(43) Pub. Date: Oct. 15, 2026

(54) FOOT PROSTHESIS COVER AND METHOD FOR DESIGNING SUCH COVER

(71) Applicant: AXILES BIONICS BV, Brussel (BE)

(72) Inventors: Pierre Philippe Cherelle, Overijse (BE); Felipe Gomez, La Hulpe (BE); Juilien De Muyter, Bruxelles (BE); Gil Lauwers, Schaerbeek (BE)

(21) Appl. No.: 19/162,833

(22) PCT Filed: Mar. 7, 2024
(86) PCT No.: PCT/EP2024/056037

§ 371 Date: Sep. 5, 2025

(30) Foreign Application Priority Data
Mar. 7, 2023 (NL) 2034277

(51) Int. Cl. A61F 2/50; A61F 2/66

(52) U.S. Cl. CPC A61F 2/5046; A61F 2/66; A61F 2002/5001; A61F 2002/5003

(57) ABSTRACT

An integral foot prosthesis cover (10) configured for at least partially covering a foot prosthesis, said integral cover comprising at least one functional portion (11a, 11b, 12, 13, 14, 17, 18) and a main cover portion; wherein said at least one functional portion has at least one functional density distribution and said main cover portion has a main density distribution; said at least one functional density distribution being different from said main density distribution; and wherein preferably the at least one functional density distribution is configured for enhancing a functionality of the at least one functional portion.

### CROSS-REFERENCE TO RELATED APPLICATIONS

This application is the US National Stage of International Application No. PCT/EP2024/056037, filed Mar. 7, 2024, which claims priority to Netherlands Patent Application No. 2034277, filed Mar. 7, 2023, which are each incorporated by reference herein in their entirety.

### FIELD OF INVENTION

The field of the invention relates to covers for prostheses, and in particular for foot prostheses, and to methods for designing prosthesis covers with functional features, and in particular foot prosthesis covers.

### BACKGROUND

In existing prosthetic foot designs, it is advantageous to approximate the feel and fluid range of motion of a human foot's natural stride, and in particular the ability to fluidly transition from heel-strike to toe-off during motion of the foot. Although the foot prosthesis itself contributes to this desire, also the foot prosthesis cover will play a role in fulfilling this function.

The foot prosthesis cover receives the foot member of the foot prosthesis, and typically has the shape of a real foot. Existing foot covers are generally made of a flexible material and have as a main purpose to cover the foot member of the prosthesis, to give the foot member the look and feel of a real foot and to interface correctly with the shape of the shoes.

### SUMMARY

The object of embodiments of the invention is to provide a foot prosthesis cover with improved properties or features and in particular a foot prosthesis cover which may help in giving a user the feel and fluid range of motion of a human foot's natural stride.

According to a first aspect of the invention, there is provided an integral foot prosthesis cover configured for at least partially covering a foot prosthesis. The integral cover comprises at least one functional portion and a main cover portion, wherein the at least one functional portion is integrally connected with the main cover portion. The at least one functional portion has at least one respective functional density distribution and the main cover portion has a main density distribution. The at least one functional density distribution is different from the main density distribution. Preferably, the at least one functional density distribution is configured for enhancing a functionality of the at least one functional portion.

By having at least one functional portion with a different density distribution as compared to the density distribution of the main cover portion, the cover can contribute to a good user experience, and in particular a user experience which provides the user with a feel of motion of a human foot's natural stride. Indeed, by including one or more functional portions in one or more appropriate locations in the cover, adapted properties may be provided by selecting a suitable density profile for those one or more portions. Suitable adapted properties may include any one or more of the following: suitable shock absorbing properties, a suitable flexibility, and in particular a suitable flexibility in the toe area, better eversion/inversion properties, suitable torsion adaption properties, improved usability, improved maintenance and/or handling of the cover, etc.

Preferably, the at least one functional density distribution is different from the main density distribution in that any one or more of the following is different: an infill ratio, an infill pattern, an average density, such as a foam density. Especially when the cover is made by 3D printing, an infill ratio and/or an infill pattern may be selected in function of the desired properties of the functional portion. Also, if a material is used which is configured for hardening at variable densities depending on e.g. the temperature, such as a foam-type polymeric material, and a functional portion may be hardened at an average density which is different from the average density of the main cover portion.

Preferably, the at least one functional portion comprises one or more of the following: a shock absorption portion, a torsion portion, a flexibility enhancement portion, an inversion/eversion portion. Preferably the shock absorption portion is configured to have enhanced shock absorption capabilities as compared to the main cover portion. Preferably, the torsion portion is configured to have enhanced torsion adaption properties as compared to the main cover portion. Preferably, the flexibility enhancement portion is configured to have an increased flexibility as compared to the main portion. Preferably, the inversion/eversion portion is configured to have enhanced inversion/eversion capabilities as compared to the main cover portion. The inversion/eversion portion may be included by a lateral functional portion located on a lateral section of the foot prosthesis cover. Different portions of the foot have different functions, and by including one or more of the functions listed above, the cover may contribute in an improved manner to a good user experience. It is noted that the different functional portions may be located adjacent to one another, or one above the other, and more generally in any possible configuration.

Preferably, the foot prosthesis cover is made in a single piece.

Preferably, the foot prosthesis cover is made at least partially, and preferably fully, by three-dimensional, 3D, printing. This process allows forming the at least one functional portion in a robust and easy manner.

Preferably, the at least one functional portion comprises at least one infill portion surrounded by material of the main cover portion. An infill portion is a portion where material is arranged according to a pattern so that the infill portion comprises empty areas and areas of solid material. The areas of solid material may comprise wall or pillar portions adjacent areas filled with air. The distribution, size and shape of such wall portions or pillar portions determine the properties of the functional portion and by choosing an appropriate infill structure appropriate properties may be given to the functional portion.

The infill portion may have a pattern which is any one of the following or a combination thereof: polygonal, such as honeycomb or triangular, rectilinear, wiggle, grid, circular, spiral, gyriod, cubic, octet, voronoi, etc. When the pattern is circular, one may think of solid pillars between two walls of a cavity or hollow pillars between two walls of a cavity. When a wiggle pattern is used, two walls of a cavity may be joined by walls having wall sections which are at an angle. When a rectilinear pattern is used, two walls of a cavity may be joined by straight walls.

Preferably, the cover comprises a sole portion and a peripheral portion extending upwardly from the sole portion. An infill portion of said at least one infill portion may be arranged between a first layer and a second layer of the sole portion. Also, an infill portion of said at least one infill portion may be arranged between a first layer and a second layer of the peripheral portion.

Optionally, the peripheral portion may comprise a lateral section of the foot prosthesis cover and the at least one functional portion may comprise a lateral functional portion located in the lateral section.

Preferably, the infill portion comprises a plurality of walls with air in between the plurality of walls, extending between two layers of the main cover portion. For example, the at least one functional portion may comprise a torsion infill portion with curved walls, said torsion infill portion extending in the sole portion of the cover, preferably in an area surrounding a pylon axis of the foot prosthesis. More specifically, the curved walls connect mechanically in a vertical manner the two layers (inner and outer) of the main cover portion in order to link their motions, one relative to the other. In this way the two layers of the main cover portion will be able to move with respect to each other, resulting in improved torsion properties. In addition or alternatively, the at least one functional portion may comprise an inversion/eversion infill portion, said inversion/eversion infill portion extending in the sole portion of the cover. In this way, some rotational movement around a longitudinal center line is made possible, resulting in improved inversion/eversion handling capabilities. The inversion/eversion infill portion may comprise cavities which vary in shape and/or size, e.g. increasing is size as the distance of the cavity with respect to the longitudinal center line of the sole increases. In an alternative embodiment, the inversion/eversion portion is not an infill portion, but is a portion with a varying density, wherein the density decreases, e.g., in a continuous or stepwise manner, as the distance with respect to the longitudinal center line of the sole increases

Preferably, the at least one functional portion comprises a back heel functional portion located at a back heel section of the foot prosthesis cover. Preferably, the back heel functional portion is a shock absorption portion. In this way, shocks occurring during a heel strike may be dampened in an appropriate manner.

Preferably, the at least one functional portion comprises a toe functional portion located at a toe section of the foot prosthesis cover and/or a ball functional portion located at a ball section of the foot prosthesis cover. Preferably, the toe and/or ball functional portion is a flexibility enhancement portion. In this way. In this way, the necessary flexibility may be provided during a toe-strike and during movement in general.

Preferably, the at least one functional portion comprises a plantar heel functional portion located at a plantar heel section of the foot prosthesis, wherein preferably said plantar heel functional portion is a torsion portion. In this way, shear stress at the stump in trans-tibial amputees induced by turning movements may be reduced with the use of such torsion portion.

Optionally, the at least one functional portion comprises a heel functional portion located at an upper heel section of the foot prosthesis, for example between the heel and an upper edge of the cover. By giving additional flexibility to such heel functional portion, and improved handling of the cover may be achieved. More in particular the arranging of the cover over the foot prosthesis may be done in an easier manner. Additionally or alternatively, the at least one functional portion may comprise a front top functional portion located in the peripheral portion of the cover, between a top front section and the upper edge of the peripheral portion of the cover, i.e. in a location diametrically opposite of the above described heel functional potion with respect to the upper edge of the cover. Providing such top front functional portion also allows to arrange the cover over the foot prosthesis in an easier manner.

In a possible embodiment, the cover is made of a foam-type polymeric material configured for hardening at variable densities, e.g. because of a different temperature, and wherein the at least one functional portion comprises a functional portion which has been hardened at a density which is different from the density of the main cover portion.

Preferably, the at least one functional portion and the main cover portion are made of the same material. Especially when the cover is made by 3D printing, the process can be simple and effective. However, in some embodiments it is possible to use different materials. For example, the at least one functional portion may comprise a functional portion made of a material which is different from a material used for the main cover portion.

Optionally, the at least one functional portion comprises a functional portion made of multiple layers having different density distributions. In this manner the functional portion can be provided with properties which are even more tailored to the needs of the user.

Optionally, the at least one functional density distribution of the at least one functional portion has one or more characteristics, e.g. an infill ratio, an infill pattern, an average density, varying within a predetermined range. In this way, the difference between the main cover portion and the at least one functional portion may be designed to be progressive with a continuous change in a defining parameter of one or more of the infill ration, the infill patter, and the average density. Also, the mechanical response engineered by having such functional portion may be refined in such a way.

Optionally, the foot prosthesis cover has a sole portion with an inner face provided with a recess configured to accommodate a fixation means when the foot prosthesis cover is affixed to the foot prosthesis, without protruding out from the foot prosthesis cover.

The foot prosthesis cover may be made of any suitable material, e.g. a thermoplastic elastomer (TPE), such as a thermoplastic polyurethane (TPU). Preferred materials are foaming TPE's, such as foaming TPU's, and in particular materials suitable for 3D printing.

According to another aspect of the invention, there is provided a method for designing a foot prosthesis cover for a user, said foot prosthesis cover comprising at least one functional portion. The method comprises the steps of obtaining user information such as any one or more of the following: a total weight and/or height of the user, foot prosthesis information about the foot prothesis of the user, a user preference, information related to an impact level, information related to a level of activity of the user; based on the obtained user information, determining at least one parameter of the at least one functional portion of the foot prosthesis cover; forming said foot prosthesis cover based on said at least one determined parameter.

Using such a method the foot cover may be tailored to the needs of the user. More in particular, any functional portion of the foot cover may be adapted to the needs of the user by determining suitable properties, such as a suitable density distribution, for the functional portion.

Technical advantages explained above for the foot prosthesis cover apply mutatis mutandis for the method. Further, the method may be used to manufacture any one of the embodiments of the foot prosthesis cover set out above.

Preferably, the at least one parameter comprises one or more of the following: at least one parameter representative for a density distribution of the at least one functional portion, at least one dimension parameter and/or shape parameter of the at least one functional portion. More generally any parameter characterizing a functional portion may be determined. In some embodiments, a functional portion may have a fixed shape and/or fixed dimensions, and only a parameter representative for a density distribution of the at least one functional portion may be determined, while in other embodiments, the density distribution may be fixed and a suitable dimension and/or shape may be determined. In yet other embodiments, a shape parameter and a dimension parameter and a density related parameter are determined.

Preferably, the step of forming comprising printing said foot prosthesis cover using 3D printing.

Preferably, the step of forming comprises using an infill structure for a functional portion of said at least one functional portion. Preferably no infill structure is used in a main cover portion. A property of the infill structure may be determined by said at least one parameter. For example, the at least one parameter may relate to the infill pattern and/or to the infill ratio to be used for the functional portion.

Preferably, the step of forming is performed such that the at least one functional density distribution is different from a main density distribution of a main cover portion of the cover in that any one or more of the following is different: an infill ratio, an infill pattern, an average density, such as a foam density. In other words, the at least one parameter may specify e.g. an infill ratio and/or an infill pattern and/or an average density to be used for each functional portion.

Preferably, the at least one functional portion comprises one or more of the following: a shock absorption portion, a torsion portion, a flexibility enhancement portion, an inversion/eversion portion. For example, the at least one functional portion may comprise a back heel functional portion located at a back heel section of the foot prosthesis cover, and the step of determining may comprise determining at least one parameter representative for a shock absorption property of the back heel functional portion. Also, the at least one functional portion may comprise a toe functional portion located at a toe section of the foot prosthesis cover and/or a ball functional portion located at a ball section of the foot prosthesis cover, and the step of determining may comprise determining at least one parameter representative for a flexibility of the toe and/or ball functional portion. Further, the at least one functional portion may comprise a plantar heel functional portion located at a plantar heel section of the foot prosthesis, and the step of determining may comprise determining at least one parameter representative for a torsion adaption property of the plantar heel functional portion. Further, the at least one functional portion may comprise an inversion/eversion functional portion, and the step of determining may comprise determining at least one parameter representative for a inversion/eversion property.

Optionally, the step of forming comprises forming the cover using a foam-type polymeric material configured for hardening at variable densities, wherein a functional portion of said at least one functional portion is formed at a density which is different from the density of a main cover portion.

Preferably, the step of forming comprises forming the entire cover of the same material.

According to a further aspect of the invention, there is provided a computer program comprising computer-executable instructions to perform one or more steps of any one of the embodiments of the method disclosed above, when the program is run on a computer. According to a further aspect of the invention, there is provided a computer device or other hardware device programmed to perform one or more steps of any one of the embodiments of the method disclosed above. According to another aspect there is provided a data storage device encoding a program in machine-readable and machine-executable form to perform one or more steps of any one of the embodiments of the method disclosed above.

Whilst the embodiments above have been described for a foot prosthesis, it will be understood that embodiments of the invention also relate to covers for other prosthesis or for orthosis or for exoskeletons. So generally, the invention relates to an integral cover configured for at least partially covering a prosthesis, orthosis or exoskeleton, said integral cover comprising at least one functional portion and a main cover portion; wherein said at least one functional portion has at least one functional density distribution and said main cover portion has a main density distribution; said at least one functional density distribution being different from said main density distribution; and wherein preferably the at least one functional density distribution is configured for enhancing a functionality of the at least one functional portion. Any of the preferred features described above for a cover of a foot prosthesis may also be included in a cover for at least partially covering any prosthesis, orthosis or exoskeleton.

Also, the method disclosed above may be used more generally for designing a cover for a prosthesis, orthosis or exoskeleton, said cover having at least one functional portion. The method comprises the following steps:
obtaining information related to a user of the prosthesis or orthosis or information related to the exoskeleton;
based on the obtained information, determining at least one parameter for characterizing the at least one functional portion of the cover;
forming said cover based on said at least one determined parameter.

Any of the preferred features described above for the method of designing a cover of a foot prosthesis may also be included in a method for designing a cover for at least partially covering any prosthesis, orthosis or exoskeleton.

### BRIEF DESCRIPTION OF THE DRAWINGS

This and other aspects of the present invention will now be described in more detail, with reference to the appended drawings showing a currently preferred embodiment. Like numbers refer to like features throughout the drawings.

FIG. 1A illustrates a schematic cut side view of an exemplary embodiment of a foot prosthesis cover.

FIG. 1B depicts a schematic bottom view of the exemplary embodiment of FIG. 1A.

FIGS. 2A-2C illustrate schematically different infill structures which may be used in exemplary embodiments of the invention.

FIGS. 3A and 3B illustrate a schematic section through a sole and a schematic cross section of an exemplary embodiment of a cover with a heel shock absorption portion and toe flexibility portions.

FIGS. 3C and 3B illustrates two schematic side views of exemplary embodiments of a cover with a back heel functional portion.

FIGS. 4A and 4B illustrate a schematic section through a sole and a schematic cross section of an exemplary embodiment of a cover with a torsion functional portion.

FIGS. 5A and 5B illustrate a schematic section through a sole and a schematic cross section of another exemplary embodiment of a cover with a torsion functional portion.

FIGS. 6A and 6B illustrate a schematic section through a sole and a schematic cross section of another exemplary embodiment of a cover with an inversion/eversion functional portion.

FIG. 7 illustrates different possible infill patterns which may be used in embodiments of the invention.

FIGS. 8A and 8B illustrate schematic side views of a cover arranged around a foot prosthesis, wherein the cover of FIG. 8B has been made transparent to reveal the foot prosthesis.

FIG. 9 is a flow chart illustrating an exemplary embodiment of a method for designing a foot prosthesis cover for a user.

### DETAILED DESCRIPTION OF EMBODIMENTS

For the sake of simplicity, embodiments related to a cover for a foot prosthesis with an ankle joint system for a human subject will be detailed below. However, the skilled person will understand that the disclosed features below can be freely adapted to an animal subject or for another limb, for example.

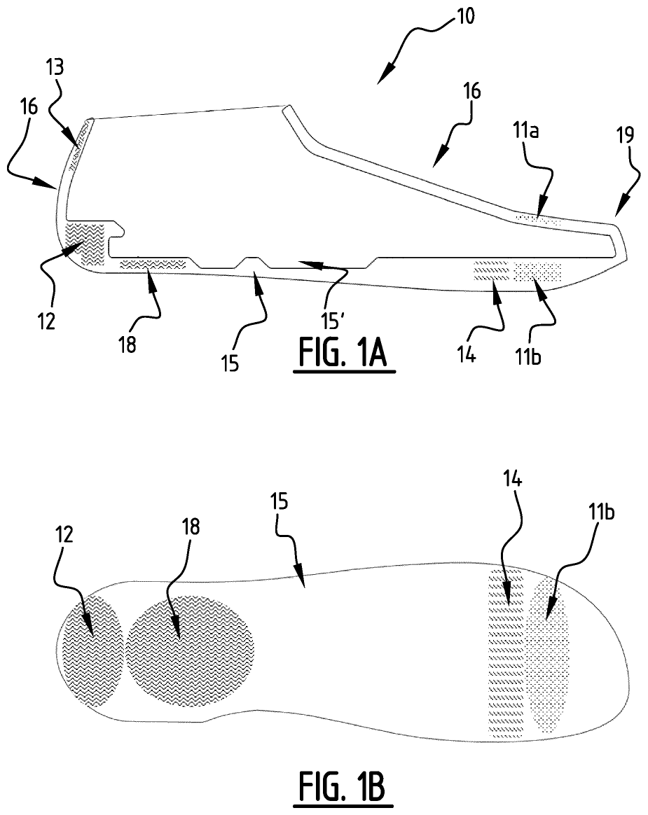

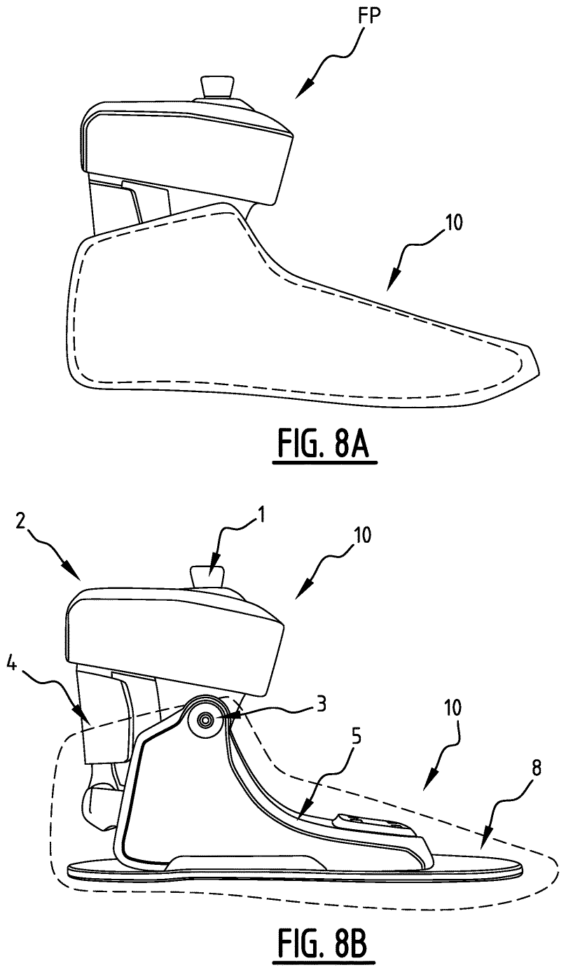

FIGS. 1A and 1B illustrate an integral foot prosthesis cover 10 configured for at least partially covering a foot prosthesis. FIGS. 8A and 8B illustrate how the cover 10 can be arranged around a foot prosthesis FP. Typically a foot prosthesis comprises a foot structure 5 with a basis 8, a leg part 2 with an adapter 1, a spring element or actuator 4 between the foot structure 5 and the leg part 2, an ankle pivot 3 between the foot structure 5 and the leg part 2.

The cover 10 comprises a sole portion 15 and a peripheral portion 16 extending upward from the sole portion 15. An outer surface of the cover may have more or less the shape of a foot and is adapted to cover the foot prosthesis FP. The cover 15 may have a toe portion 19 with or without distinct toes. Additionally, the peripheral portion 16 may comprise a lateral section, and the at least one functional portion may comprise a lateral functional portion located in the lateral section.

The integral foot prosthesis cover 10 comprises a plurality of functional portions 11a, 11b, 12, 13, 14, 18 and a main cover portion (not indicated with a reference numeral but this is the portion of the cover 10 which is not a functional portion). In FIGS. 1A and 1B the functional portions are indicated schematically with a line pattern and the main cover portion is indicated as a solid white portion. It is noted that FIG. 1A shows five functional portions 11a, 11b, 12, 13, 14, 18, but a cover of the invention may also include one, two, three or four functional portions or even more than five functional portions.

The functional portions 11a, 11b, 12, 13, 14, 18 have respective functional density distributions and the main cover portion has a main density distribution. The respective functional density distributions are different from the main density distribution. Preferably, the respective functional density distributions are configured for enhancing the respective functionalities of the functional portions 11a, 11b, 12, 13, 14, 18.

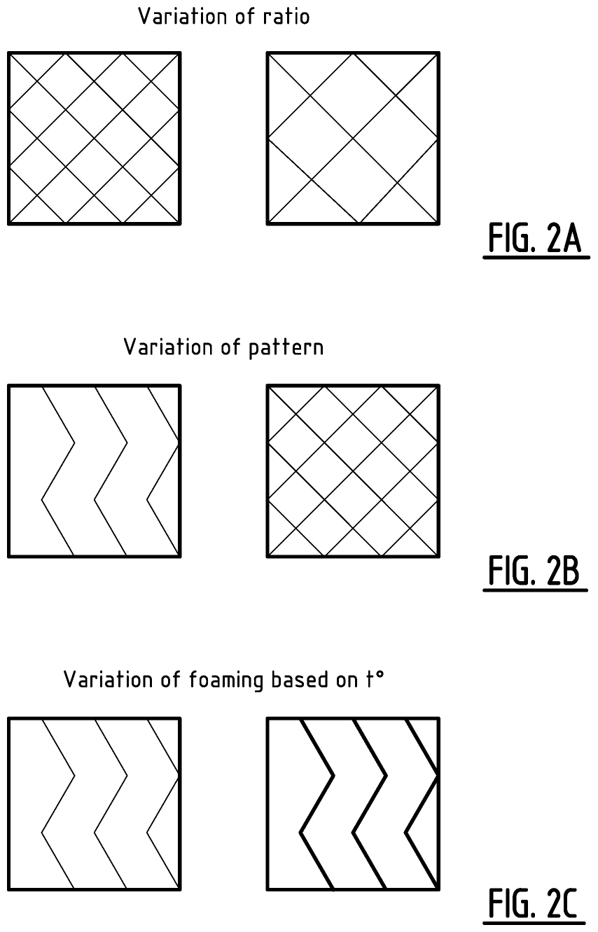

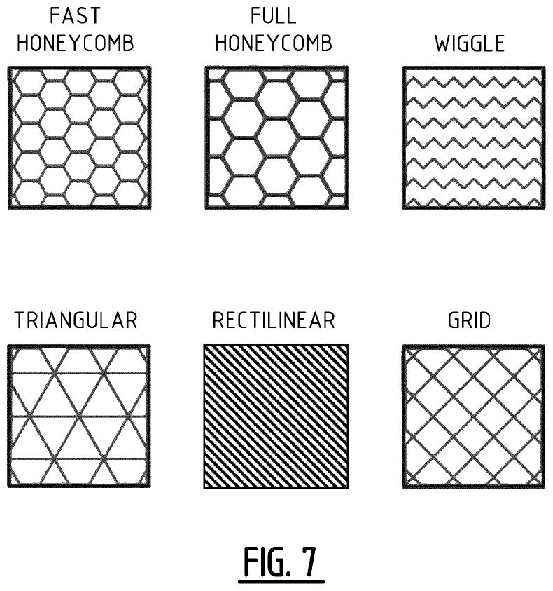

Preferably, the respective functional density distributions are each different from the main density distribution in that any one or more of the following is different: an infill ratio, an infill pattern, an average density, such as a foam density. FIG. 2A illustrates two different infill ratios resulting in different properties. It is noted that an infill ratio of the main portion may typically be 100%, i.e. a solid material area, whilst the infill ratio of the functional parts 11a, 11b, 12, 13, 14, 18 may be lower than 100%. FIG. 2B illustrates two different infill patterns, here a wiggle pattern on the left and a grid pattern on the right, resulting in different properties. FIG. 2C illustrates the result of using a different temperature during hardening of a foam material, resulting in different average densities. FIG. 7 illustrates some possible infill patterns that may be used in embodiments of the invention: honeycomb, wiggle, triangular, rectilinear, grid. The skilled person understand that many other options exist, such as circular (hollow or solid), etc. Typically, the infill structure extends between two opposite walls of a cavity of the main cover portion. The infill structure may comprise walls, e.g. honeycomb walls extending between two opposite walls of the cavity. In another embodiment the infill structure may comprise pillars (solid or hollow) extending between two opposite walls of the cavity.

Optionally, a functional portion may be made of multiple layers having different density distributions, e.g. different infill patterns and/or ratios.

Preferably, the functional portions comprise a back heel functional portion 12 located at a back heel section of the foot prosthesis cover. Preferably, the back heel functional portion 12 is a shock absorption portion. Preferably, the shock absorption portion 12 is configured to have enhanced shock absorption capabilities as compared to the main cover portion.

Preferably, the functional portions comprise a toe functional portion 11a located at a toe section 19 of the foot prosthesis cover 10 and/or a ball functional portion 11b located at a ball section of the foot prosthesis cover 10. Preferably, the toe and/or ball functional portion 11a, 11b is a flexibility enhancement portion. Preferably, the flexibility enhancement portion 11a, 11b is configured to have an increased flexibility as compared to the main portion.

Preferably, the functional portions comprise a torsion functional portion 18 located for example at a plantar heel section of the foot prosthesis. It is noted that this torsion functional portion 18 may extend over a large section of the plantar side of the foot prosthesis, preferably mainly covering an area corresponding with the plantar aponeurosis. Preferably, the torsion functional portion 18 is located below the pylon axis P of the foot prosthesis. Preferably, the torsion portion 18 is configured to have enhanced torsion adaption properties as compared to the main cover portion.

Optionally, the functional portions comprise a further shock absorbing portion 14 in the front sole portion of the cover 10, which may help in absorbing shocks when descending from stairs, and/or a further flexibility enhancement heel portion 13. Such flexibility enhancement heel portion 13 may result in an easier handling of the cover 10. Additionally or alternatively, the at least one functional portion may comprise a front top functional portion (not shown) located in the peripheral portion of the cover, between a top front section and an upper edge 16a of the peripheral portion of the cover, i.e. in a location diametrically opposite of the above described heel functional potion 13 with respect to the upper edge of the cover 16a.

In the embodiment of FIGS. 1A and 1B multiple functional portions are indicated, but the skilled person understands that also a single functional portion or any combination of functional portions may be included, e.g. only portion 18, or only portion 11b, or only portion 11a, or only the combination of 11a and 11b, or only portion 12, or only portion 13, or only portion 14.

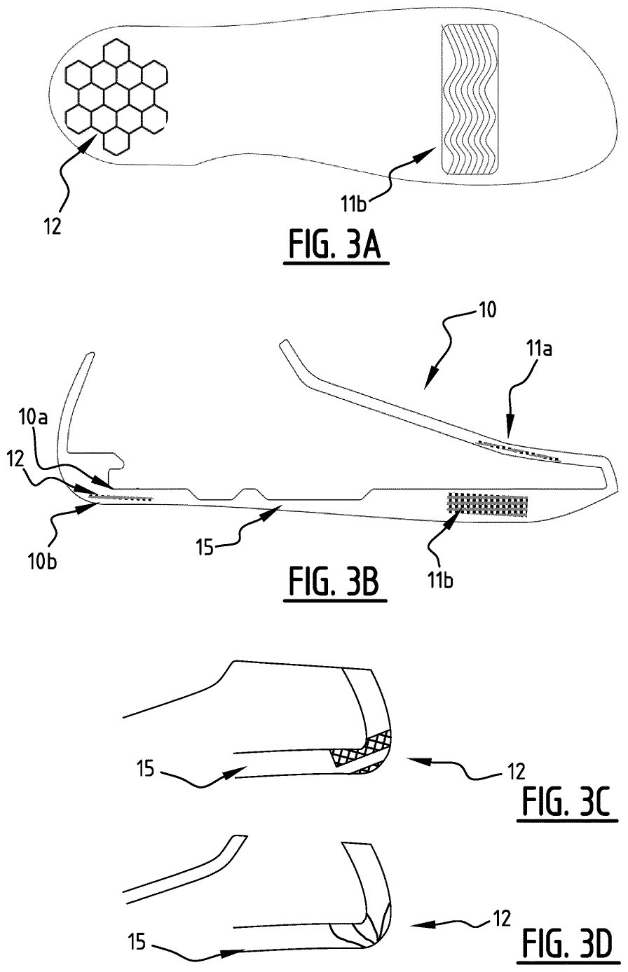

FIGS. 3A and 3B illustrate another exemplary embodiment of a cover 10 with a back heel functional portion 12 and toe portions 11a, 11b. The back heel functional portion 12 may comprise a single infill layer or multiple infill layers. The infill portion may have a honeycomb pattern or any other suitable pattern capable of improving the shock absorption properties. In a further developed embodiments, multiple infill layers 12 with a different infill ratio may be included, see FIG. 3C. Also, the back heel functional portion 12 may comprise an optimized infill pattern, see FIG. 3D. The toe functional portion 11a and the ball functional portion 11b may have e.g. a low infill ratio and/or be hardened at a low temperature. Any one or more of the above described other functional portions 13, 14, 18 may also be added in this embodiment.

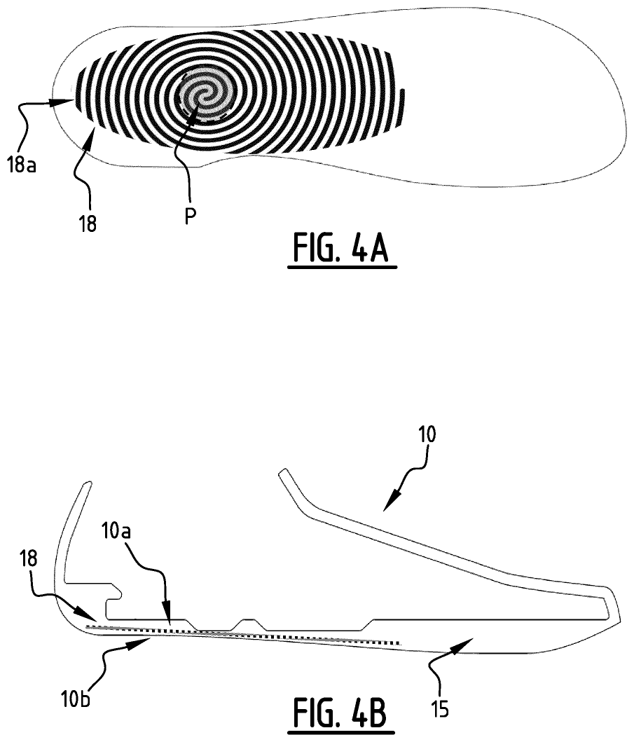

FIGS. 4A-4B illustrate another exemplary embodiment of a cover 10 with a torsion functional portion 18 in more detail. The torsion functional portion 18 comprises an infill portion with a spiral-like pattern. The infill portion 18 is located within the sole 15 of the cover, between a top layer 10a and a bottom layer 10b of main cover material. Optionally, the infill portion extends at least below the pylon axis P of the foot prosthesis, as illustrated in FIG. 4A. Alternatively, the portion below the pylon axis may be a solid portion and the infill portion may surround the solid portion. The infill portion 18 comprises curved walls, and in particular spiral-like-shaped walls 18a, extending between the bottom layer 10b and the top layer 10a of the sole 15 of the cover 10. Preferably, a center of the spiral-like shaped walls is located below the pylon axis P of the foot prosthesis. By including such torsion functional portion 18, layer 10a will be able to move, e.g., rotate around pylon axis P and/or translate with respect to the bottom layer 10b of the sole 15, resulting in good torsion adaptation properties. Any one or more of the above-described other functional portions 11a, 11b, 12, 13, 14 may also be added in this embodiment.

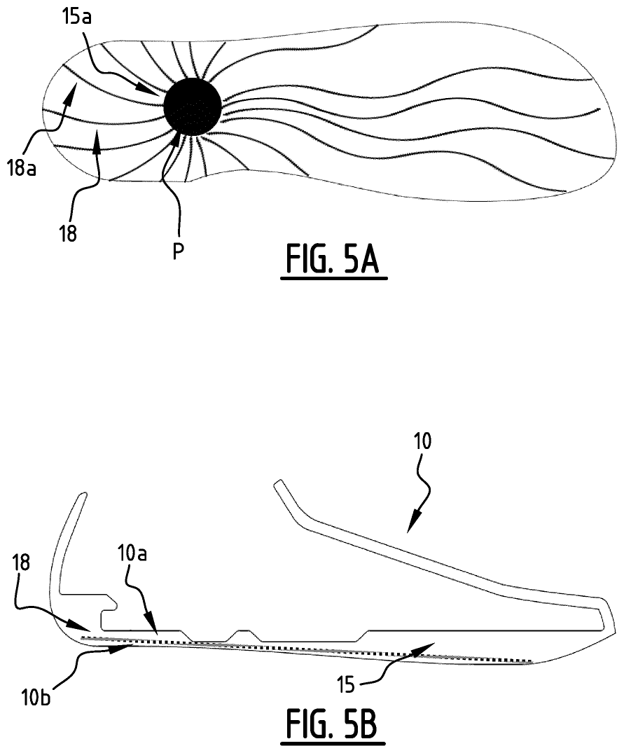

FIGS. 5A-5B illustrate another exemplary embodiment with a torsion functional portion 18 in more detail. The torsion functional portion 18 comprises an infill portion with a wavy pattern. The infill portion 18 is located within the sole 15 of the cover 10, between a top layer 10a and a bottom layer 10b of main cover material. As illustrated, the infill portion surrounds a solid portion 15a located below the pylon axis P of the foot prosthesis. Torsion portion 18 may extend across substantially the entire sole 15, e.g. across at least 75% of the surface of the sole 15, between a bottom layer 10b and top layer 10a of the sole 15. The infill portion 18 comprises curved walls 18a, and in particular wavy walls, extending between the bottom layer 10b and the top layer 10a of the sole 15 of the cover 10, from a solid area 15a below the pylon axis P in the direction of the edges of the sole 15. By including such torsion functional portion 18, layer 10a will be able to move, e.g. rotate around pylon axis P and/or translate with respect to the bottom layer 10b of the sole 15, resulting in good torsion adaptation properties. Any one or more of the above-described other functional portions 11a, 11b, 12, 13, 14 may also be added in this embodiment.

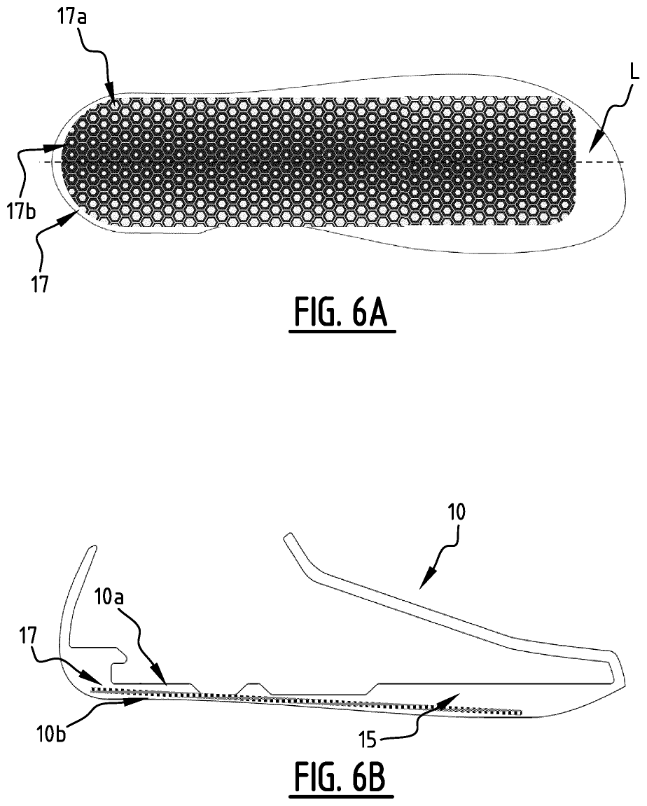

FIGS. 6A-6B illustrate another exemplary embodiment with an inversion/eversion functional portion 17. The inversion/eversion functional portion 17 comprises an infill portion with cavities 17a, 17b which vary in volume. The infill portion 17 is located within the sole 15 of the cover 10, between a top layer 10a and a bottom layer 10b of main cover material. The cavities 17a which are further away from a longitudinal center line L are larger in size than the cavities 17b which are closer to the longitudinal center line than the cavities 17a. More generally, it is preferred that the density of the inversion/eversion portion 17 decreases (in a continuous or discontinuous manner) as the distance of the longitudinal center line L increases. In this way, some rotation about axis L is possible, resulting in an inversion/eversion movement. Inversion/eversion functional portion 17 may extend across a large portion of the sole 15, e.g. across at least 70% of the surface of the sole 15, between a bottom layer 10b and top layer 10a of the sole 15, but may also extend across a smaller portion of the sole 15. The infill portion 17 comprises cavities 17a, 17b, e.g., hexagonal cavities, separated by walls, extending between the bottom layer 10b and the top layer 10a of the sole 15 of the cover 10. Any one or more of the above-described other functional portions 11a, 11b, 12, 13, 14, 18 may also be added in this embodiment. For example, a torsion portion 18 may be located below or above the inversion/eversion portion 17. More generally, functional portions may be arranged next to each other, and/or one above the other. Additionally, although not shown, an additional or alternative inversion/eversion portion may be included in a lateral functional portion located at a lateral section of the foot prosthesis cover.

Preferably, the foot prosthesis cover 10 is made in a single piece. The foot prosthesis cover may be made at least partially, and preferably fully, by three-dimensional, 3D, printing. Optionally, the cover is made of a foam-type polymeric material configured for hardening at variable densities, and one or more functional portions are hardened at a density which is different from the density of the main cover portion. Preferably, the functional portions 11a, 11b, 12, 13, 14, 17, 18 and the main cover portion are made of the same material. However, in some embodiments, one or more functional portions may be made of a material which is different from a material used for the main cover portion.

The foot prosthesis cover has a sole portion 15 with an inner face provided with a recess 15′ configured to accommodate a fixation means (not shown) when the foot prosthesis cover 10 is affixed to the foot prosthesis.

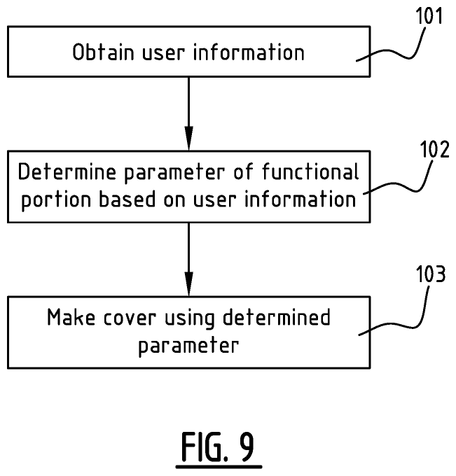

FIG. 9 illustrates a method for designing a foot prosthesis cover for a user, for example any one of the foot prosthesis cover embodiments disclosed above. The method comprises the following steps:
obtaining user information, see step 101, such as one or more of the following: a total weight and/or height of the user, foot prosthesis information about the foot prothesis, a user preference, impact level information, activity information;
based on the obtained user information, determining at least one parameter of the at least one functional portion of the foot prosthesis cover, for example at least one parameter representative for at least one functional density distribution of the at least one functional portion of the foot prosthesis cover, see step 102;
forming said foot prosthesis cover based on said at least one determined parameter, see step 103.

Steps 101 and 102 may be performed by a computer program. For example, in order to obtain the user information, the user may be presented with a user information input screen for inputting the user information. In addition or alternatively, user information may be obtained by collecting information, such a sensor information, while a user is carrying the foot prosthesis, during different activities such as walking, running, standing, etc. Based on the obtained user information, parameters to characterize one or more functional portions may be determined. Such parameters may relate to the functional density distribution (e.g. infill pattern, infill ratio, density, etc.), to the shape and dimension of the functional portion, and more generally to any property of the of a functional portion to be included in the cover.

The step 103 of making the cover, may comprise printing the foot prosthesis cover using 3D printing. The step of forming 103 may comprise using an infill structure for a functional portion (e.g. any one or more of functional portions 11a, 11b, 12, 13, 14, 17, 18 described above), wherein a property of said infill structure is determined by the at least one parameter determined in step 102.

The step 103 of making the cover may be performed such that the at least one functional density distribution is different from a main density distribution of a main cover portion of the cover in that any one or more of the following is different: an infill ratio, an infill pattern, an average density, such as a foam density.

The step 103 of forming the cover may comprise forming the cover using a foam-type polymeric material, such as foaming TPU, configured for hardening at variable densities, wherein a functional portion of said at least one functional portion is formed at a density which is different from the density of a main cover portion. The step 103 may comprise forming the entire cover of the same material.

Whilst the principles of the invention have been set out above in connection with specific embodiments, it is to be understood that this description is merely made by way of example and not as a limitation of the scope of protection which is determined by the appended claims.

1. An integral foot prosthesis cover configured for at least partially covering a foot prosthesis, said integral cover comprising:
at least one functional portion and a main cover portion;
wherein said at least one functional portion has at least one functional density distribution and said main cover portion has a main density distribution; said at least one functional density distribution being different from said main density distribution; and
wherein preferably the at least one functional density distribution is configured for enhancing a functionality of the at least one functional portion.

2. The foot prosthesis cover according to claim 1:
wherein the at least one functional density distribution is different from the main density distribution in that any one or more of the following is different: an infill ratio, an infill pattern, an average density, such as a foam density; and/or
wherein the at least one functional portion comprises one or more of the following: a shock absorption portion, a torsion portion, a flexibility enhancement portion, an inversion/eversion portion, wherein preferably the shock absorption portion is configured to have enhanced shock absorption capabilities as compared to the main cover portion, wherein preferably the torsion portion is configured to have enhanced torsion adaption properties as compared to the main cover portion, wherein preferably the flexibility enhancement portion is configured to have an increased flexibility as compared to the main portion, wherein preferably the inversion/eversion portion is configured to have enhanced inversion/eversion capabilities as compared to the main cover portion.

3. (canceled)

4. The foot prosthesis cover according to claim 1, wherein the foot prosthesis cover is made in a single piece, and/or wherein the foot prosthesis cover is made at least partially, and preferably fully, by three-dimensional printing.

5. (canceled)

6. The foot prosthesis cover according to claim 1:
wherein said at least one functional portion comprises at least one infill portion, and
wherein an infill portion of said at least one infill portion is preferably entirely surrounded by material of the main cover portion.

7. (canceled)

8. The foot prosthesis cover of claim 1, wherein the cover comprises a sole portion and a peripheral portion extending upwardly from the sole portion.

9. The foot prosthesis cover of claim 6:
wherein the cover comprises a sole portion and a peripheral portion extending upwardly from the sole portion;
wherein an infill portion of said at least one infill portion is arranged between a first layer and a second layer of the sole portion, and/or wherein an infill portion of said at least one infill portion is arranged between a first layer and a second layer of the peripheral portion, and/or.

10. (canceled)

11. The foot prosthesis cover according to claim 6:
wherein the infill portion has a pattern which is any one of the following or a combination thereof: polygonal, such as honeycomb or triangular, rectilinear, wiggle, grid, circular, spiral-like, gyroid, Voronoi, cubic, octet; and/or
wherein the infill portion comprises a plurality of walls with air in between the plurality of walls, extending between two layers of the main cover portion.

12. (canceled)

13. The foot prosthesis cover according to claim 8:
wherein the infill portion comprises a plurality of walls with air in between the plurality of walls, extending between two layers of the main cover portion; and
wherein the at least one functional portion comprises a torsion infill portion with curved walls, said torsion infill portion extending in the sole portion of the cover, preferably in an area surrounding a pylon axis of the foot prosthesis, or wherein the at least one functional portion comprises an inversion/eversion infill portion, said inversion/eversion infill portion extending in the sole portion of the cover and preferably comprising cavities with a varying size and/or cavity density and/or shape.

14. The foot prosthesis cover according to claim 1:
wherein the at least one functional portion comprises a back heel functional portion located at a back heel section of the foot prosthesis cover, wherein preferably said back heel functional portion is a shock absorption portion; and/or
wherein the at least one functional portion comprises a toe functional portion located at a toe section of the foot prosthesis cover and/or a ball functional portion located at a ball section of the foot prosthesis cover, wherein preferably said toe and/or ball functional portion is a flexibility enhancement portion; and/or
wherein the at least one functional portion comprises a plantar heel functional portion located at a plantar heel section of the foot prosthesis, wherein preferably said plantar heel functional portion is a torsion portion.

15-16. (canceled)

17. The foot prosthesis cover according to claim 1:
wherein the cover comprises a sole portion and a peripheral portion extending upwardly from the sole portion;
wherein the at least one functional portion comprises a top heel functional portion located in the peripheral portion of the cover, between a heel section and an edge of the peripheral portion of the cover, and/or a top front functional portion located in the peripheral portion of the cover, between a top front section and the edge of the peripheral portion of the cover.

18. (canceled)

19. The foot prosthesis cover according to claim 1, wherein the cover is made of a foam-type polymeric material configured for hardening at variable densities, and wherein the at least one functional portion comprises a functional portion which has been hardened at a density which is different from the density of the main cover portion.

20. The foot prosthesis cover according to claim 1:
wherein the at least one functional portion comprises a functional portion made of multiple layers having different density distributions; and/or
wherein the at least one functional portion and the main cover portion are made of the same material, or wherein the at least one functional portion comprises a functional portion made of a material which is different from a material used for the main cover portion.

21-22. (canceled)

23. The foot prosthesis cover according to claim 1:
wherein the foot prosthesis cover has a sole portion with an inner face provided with a recess configured to accommodate a fixation means when the foot prosthesis cover is affixed to the foot prosthesis, without protruding out from the foot prosthesis cover; and/or
wherein the foot prosthesis cover is made of a thermoplastic elastomer.

24. (canceled)

25. A method for designing a foot prosthesis cover for a user, said foot prosthesis cover having at least one functional portion, comprising the following steps:
obtaining user information;
based on the obtained user information, determining at least one parameter for characterizing the at least one functional portion of the foot prosthesis cover, wherein the at least one parameter preferably comprises one or more of the following: at least one parameter representative for a density distribution of the at least one functional portion, at least one dimension parameter and/or shape parameter of the at least one functional portion;
forming said foot prosthesis cover based on said at least one determined parameter.

26. (canceled)

27. The method according to claim 25:
wherein the step of forming comprising printing said foot prosthesis cover using 3D printing; and/or
wherein the step of forming comprises using an infill structure for a functional portion of said at least one functional portion, preferably using no infill structure in a main cover portion, wherein a property of said infill structure is determined by said at least one parameter.

28. (canceled)

29. The method according to claim 25:
wherein the user information comprises one or more of the following: a weight of the user, a height of the user, foot prosthesis information about the foot prothesis of the user, a user preference, impact level information, activity information; and/or
wherein the step of forming is performed such that the at least one functional density distribution is different from a main density distribution of a main cover portion of the cover in that any one or more of the following is different: an infill ratio, an infill pattern, an average density, such as a foam density.

30. (canceled)

31. The method according to claim 25:
wherein the at least one functional portion comprises one or more of the following: a shock absorption portion, a torsion portion, a flexibility enhancement portion, an inversion/eversion portion; and/or
wherein the at least one functional portion comprises a back heel functional portion located at a back heel section of the foot prosthesis cover, and the step of determining comprises determining at least one parameter representative for a shock absorption property of the back heel functional portion; and/or
wherein the at least one functional portion comprises a toe functional portion located at a toe section of the foot prosthesis cover and/or a ball functional portion located at a ball section of the foot prosthesis cover, and the step of determining comprises determining at least one parameter representative for a flexibility of the toe and/or ball functional portion; and/or
wherein the at least one functional portion comprises a plantar heel functional portion located at a plantar heel section of the foot prosthesis, and the step of determining comprises determining at least one parameter representative for a torsion adaption property of the plantar heel functional portion.

32-34. (canceled)

35. The method according to claim 25:
wherein the step of forming comprises forming the cover using a foam-type polymeric material configured for hardening at variable densities, wherein a functional portion of said at least one functional portion is formed at a density which is different from the density of a main cover portion; and/or
wherein the step of forming comprises forming the entire cover of the same material.

36. (canceled)

37. A digital data storage medium encoding a machine-executable program of instructions to perform any one of the steps of the method according to claim 25.

38. The foot prosthesis cover according to claim 1, wherein the at least one functional portion comprises a lateral functional portion located at a lateral section of the foot prosthesis cover, wherein preferably said lateral functional portion is an inversion/eversion portion, wherein the at least one functional distribution of the at least one functional portion has preferably one or more characteristics varying within a predetermined range.

39. (canceled)
